(19) United States
(12) Patent Application Publication
(10) Pub. No.: US 2020/0288700 A1
(43) Pub. Date: Sep. 17, 2020

(54) STEM CELL PREPARATIONS AND APPLICATION IN THE PREPARATION OF DRUGS FOR THE TREATMENT OF OSTEOARTHRITIS

(71) Applicant: GUANGZHOU SALIAI STEM CELL SCIENCE AND TECHNOLOGY CO., LTD, Guangdong (CN)

(72) Inventors: Haijia CHEN, Guangdong (CN); Xiaohu GE, Guangdong (CN); Xiaoyan WANG, Guangdong (CN); Xuejia LI, Guangdong (CN); Yanyan MA, Guangdong (CN)

(73) Assignee: GUANGZHOU SALIAI STEM CELL SCIENCE AND TECHNOLOGY CO., LTD, Guangdong (CN)

(21) Appl. No.: 16/693,390

(22) Filed: Nov. 25, 2019

Related U.S. Application Data
(63) Continuation of application No. PCT/CN2019/106187, filed on Oct. 28, 2019.

(30) Foreign Application Priority Data
Mar. 11, 2019 (CN) 201910179832.7

(51) Int. Cl. A01N 1/02; C12N 5/0775; A61K 35/51; A61P 19/02

(52) U.S. Cl. CPC A01N 1/0221; A61P 19/02; A61K 35/51; C12N 5/0665

(57) ABSTRACT

The present invention relates to the field of stem cell technology, and in particular to a stem cell preparation and its application in the preparation of drugs for the treatment of osteoarthritis (OA). The composition provided by the invention can significantly improve the activity of umbilical cord mesenchymal stem cells under cryopreservation conditions, and the stem cell preparation prepared therefrom has a good effect on repairing cartilage injury caused by OA, thereby playing a role in the treatment of OA.

### CROSS-REFERENCE TO RELATED APPLICATION

This application is a continuation of international PCT application serial no. PCT/CN2019/106187, filed on Oct. 28, 2019, which claims the priority benefit of Chinese application no. 201910179832.7, filed on Mar. 11, 2019. The entirety of each of the above-mentioned patent applications is hereby incorporated by reference herein and made a part of this specification.

### BACKGROUND

### Technical Field

The invention relates to the field of stem cells, in particular to stem cell preparations and theirs application in the preparation of drugs for the treatment of osteoarthritis.

### Description of Related Art

### Description of Related Art

Osteoarthritis (OA) is a degenerative joint disease that seriously affects cartilage and surrounding tissues. With the development of aging population and widespread obesity, the incidence of OA has continued to increase in recent years. OA treatment methods include drug interventions and surgical treatment. Drug interventions include acetaminophen, NSAIDs, intra-articular injection of lubricants and steroid hormones, and chondroprotective agents; surgical treatment includes artificial joint replacement and microfracture surgery, etc. These treatments have limited efficacy and can not prevent the progression of the disease. Therefore, cartilage injury repair has always been a thorny issue in clinical research.

In recent years, with the deepening of stem cell research and the widespread application of bioengineering technology, (stem) cell therapy has brought hope to OA patients, including autologous chondrocytes, autologous bone marrow mesenchymal stem cells, autologous/allogeneic fat mesenchymal stem cells and umbilical cord mesenchymal stem cells. Autologous chondrocytes have limited proliferative capacity and are limited by the severity of OA patients, with limited number of beneficiary patients. And the method for obtaining and amplifying a sufficient number of mesenchymal stem cells from autologous bone marrow/fat still has problems such as certain harm to patients, a long waiting time and difficulty in standardizing of cell products. The expansion ability of autologous/allogeneic bone marrow and adipose-derived mesenchymal stem cells is superior to that of autologous chondrocytes, but the expansion ability is still weak compared with cord blood/umbilical cord-derived mesenchymal stem cells. Cord blood-derived mesenchymal stem cell drugs currently used for the treatment of joint damage have been reported, but the curative effect is limited and the stability of the preparation is poor.

### SUMMARY

In view of the above, the technical problem to be solved by the present invention is to provide a stem cell preparation and its application in the preparation of drugs for the treatment of OA, which has a good repairing effect on cartilage injury caused by OA and has good stability.

The present invention provides a composition including 10 mg/mL to 150 mg/mL albumin, 100 nmol/L to 10 μmol pituitary adenylate cyclase activating polypeptide (PACAP), 10 μg/mL to 100 μg/mL vitamin C, 1 mg/mL to 10 mg/mL vitamin E and solvents.

Preferably, the solvents are composed of DMSO, plasmalyte A and 5 wt % glucose injection.

Preferably, in the present invention, the volume ratio of DMSO, plasmalyte A and 5 wt % glucose injection in the solvents is (5-10):(35-50):(35-50).

Preferably, in some embodiments, the composition consists of 100 mg/mL albumin, 1 μmol/L PACAP, 50 μg/mL vitamin C, 5 mg/mL vitamin E and solvents, and the volume ratio of DMSO, plasmalyte A and 5 wt % glucose injection in the solvents is 5:45:50.

Preferably, in some embodiments, the composition consists of 150 mg/mL albumin, 100 μmol/L PACAP, 100 μg/mL vitamin C, 1 mg/mL vitamin E and a solvent, and the volume ratio of DMSO, plasmalyte A and 5 wt % glucose injection in the solvents is 10:35:50.

Preferably, in some embodiments, the composition consists of 10 mg/mL albumin, 10 μmol/L PACAP, 10 μg/mL vitamin C, 10 mg/mL vitamin E and a solvent, and the volume ratio of DMSO, plasmalyte A and 5 wt % glucose injection in the solvents is 5:50:35.

The compositions of the present invention are used as a cell cryopreservation solution.

The compositions provided by the present invention are used for a cell cryopreservation solution, and can significantly improve the survival rate of cells. The cells survival rate was still 95.72% within 6 months.

The present invention also provides a method of cryopreservation of cells, which is resuspended cells in a plasmalyte A, the cell suspension is mixed with an equal volume of the composition of the present invention, and then stored in liquid nitrogen.

The frozen cell cryopreservation solution includes: 5 mg/mL to 75 mg/mL albumin, 50 nmol/L to 5 μmol/L PACAP, 5 μg/mL to 50 μg/mL vitamin C, 0.5 mg/mL to 5 mg/mL vitamin E and solvents; the solvents of the cryopreservation solution consist of DMSO, plasmalyte A and 5 wt % glucose injection, with the volume ratio of DMSO, plasmalyte A and 5 wt % glucose injection is (5-10):(130-145):(35-50).

In the embodiment with the best cryopreservation effect, the cryopreservation solution consists of 50 mg/mL albumin, 0.5 μmol/L PACAP, 25 μg/mL vitamin C, 2.5 mg/mL vitamin E and solvents. The solvents of the cryopreserved solution were composed of DMSO, plasmalyte A and 5 wt % glucose injection with a volume ratio of 5:145:50.

Preferably, the suitable cells for cryopreservation according to the present invention are umbilical cord mesenchymal stem cells.

Preferably, the compositions of the present invention are applied in the preparation of stem cell preparations.

The compositions of the present invention are useful in the preparation of stem cell preparations for the treatment of OA. The stem cell preparations can be used fresh or frozen. Studies have shown that after 6 months of cryopreservation of the stem cell preparation provided by the present invention, 95.72% of the cells survived for good cartilage repair ability.

The invention also provides a stem cell preparation including: mesenchymal stem cell and the composition of the present invention.

In the present invention, the mesenchymal stem cells are umbilical cord mesenchymal stem cells.

In the present invention, the density of the mesenchymal stem cells is 2.5×10⁶ cells/mL to 25×10⁶ cells/mL. The stem cell preparation further includes a plasmalyte A, wherein the volume ratio of the plasmalyte A and the composition is 1:1.

In some embodiments, the stem cell preparation includes 2.5×10⁶ cells/mL to 25×10⁶ cells/mL mesenchymal stem cells, 5 mg/mL to 75 mg/mL albumin, 50 nmol/L to 5 μmol/L PACAP, 5 μg/mL to 50 μg/mL vitamin C, 0.5 mg/mL to 5 mg/mL vitamin E and a solvent; the solvent is composed of DMSO, plasmalyte A and 5 wt % glucose injection, wherein the volume ratio of DMSO, plasmalyte A and 5 wt % glucose injection is (5-10):(130-145):(35-50).

In the embodiment for performing efficacy verification, the stem cell preparation includes 2.5×10⁶ cells/mL or 25×10⁶ cells/mL mesenchymal stem cells, 50 mg/mL albumin, 0.5 μmol/L PACAP, 25 μg/mL vitamin C, 2.5 mg/mL vitamin E and a solvent; the solvent is composed of DMSO, plasmalyte A and 5 wt % glucose injection with a volume ratio of 5:145:50.

The stem cell preparations of the present invention are used in the preparation of a drug to repair cartilage injury.

The stem cell preparations of the present invention are used in the preparation of a drug for treating OA.

The present invention also provides a method of repairing cartilage injury by administering stem cell preparations.

The present invention also provides a method of treating OA by administering stem cell preparations.

The composition provided by the invention can significantly improve the activity of umbilical cord mesenchymal stem cells in freezing storage conditions, and the stem cell preparations prepared by the composition can have a good role in repairing cartilage injury, thus playing a role in the treatment of OA.

### BRIEF DESCRIPTION OF THE DRAWINGS

FIG. 1A and FIG. 1B show the morphology of umbilical cord mesenchymal stem cells observed under an inverted microscope. Wherein, FIG. 1A is observed under 40 times microscope and FIG. 1B is observed under 100 times microscope.

FIG. 2 shows the flow identification of umbilical cord mesenchymal stem cells.

FIG. 3 shows the detection of differentiation potential of umbilical cord mesenchymal stem cells.

FIG. 4A to FIG. 4C show the repair effect of stem cell preparation on rabbit cartilage injury model; wherein FIG. 4 A shows left knee joint as model control group, right knee joint as sham operation group; FIG. 4B shows left knee joint as stem cell low dose treatment group 1, The right knee joint as the vehicle control group; FIG. 4C shows the left knee joint as the stem cell high dose treatment group, the right knee joint as the vehicle control group.

### DESCRIPTION OF THE EMBODIMENTS

The present invention provides a stem cell preparation and its application in the preparation of drugs for the treatment of OA, and those skilled in the art can learn from the contents of the present invention and appropriately improve the process parameters. It is to be understood that all such alternatives and modifications are obvious to those skilled in the art and are considered to be included in the present invention. The method and the application of the present invention have been described by the preferred embodiments, and it is obvious that the method and application of the present invention may be modified or combined and modified to achieve and apply the present invention without departing from the scope of the present invention. Invention technology.

The test materials used in the present invention are all commercially available products, all of which can be purchased in the market.

Umbilical cord: It is a cord-like structure connecting the mother and the fetus in the fetal period. It contains two umbilical arteries and one umbilical vein. Umbilical cord mesenchymal stem cells: There is a special embryonic mucus-like connective tissue-Huatong's gel between arteries and veins. The stromal cells isolated from Huatong's gum are human umbilical cord mesenchymal stem cells (hUC-MSCs).

Among them, the primary isolation and culture of umbilical cord mesenchymal stem cells include:

1) In a sterile environment, the umbilical cord tissue is washed with physiological saline to remove excess blood, and the outer membrane and arteriovenous tissue of the umbilical cord tissue are removed with forceps, and the obtained Huatong's rubber tissue is cut and cut to a size of 1 mm³;

2) Spread the cut tissue block into a 15 cm culture dish, and after the tissue block is fixed on the culture dish, add 20-30 ml of serum-free medium, and place it in a 37° C., 5% carbon dioxide incubator for cultivation;

3) Change the liquid twice a week to observe the growth of cells around the tissue block. After the cell confluence is longer than 80%-90%, digest the treatment and subculture.

Subculture of umbilical cord mesenchymal stem cells includes:

1) In a sterile environment, discard the medium in the culture dish and wash it with physiological saline for 2-3 times;

2) Add 3-5 ml of 0.05% trypsin for 1-2 min, observe the digestion under the microscope, and terminate the digestion with the serum-containing cell culture medium, and collect the cell suspension into the centrifuge tube;

3) Centrifuge at 500 g for 5 min at room temperature, discard the supernatant after centrifugation, add 3-5 ml serum-free medium, mix and mix thoroughly, sample and count;

4) According to the counting result, inoculate into a new culture dish at a cell density of 1×10⁴/cm²-5×10⁴/cm² and add serum-free medium, and place in a 37° C., 5% carbon dioxide incubator to continue to cultivate.

Identification of umbilical cord mesenchymal stem cells obtained above:

1) Cell morphology identification

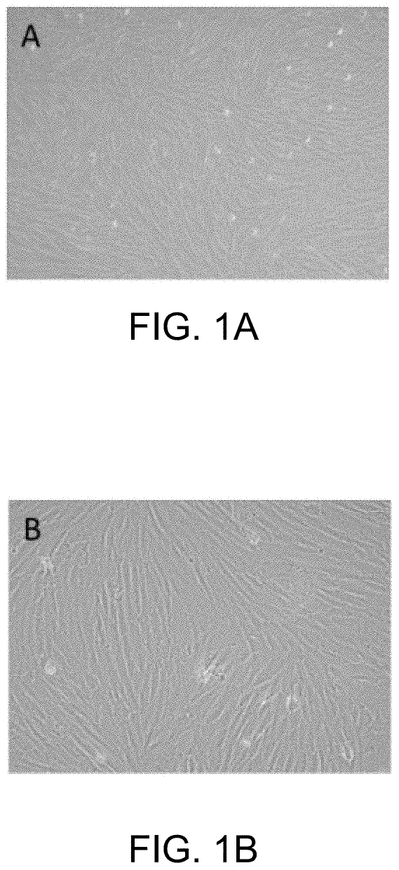

The cultured umbilical cord mesenchymal stem cells were taken and observed under an inverted microscope as shown in FIG. 1A and FIG. 1B. As shown in FIG. 1A and FIG. 1B, the cells are uniform in shape and size, and have strong refractive index. They are arranged in a long fusiform shape and conform to the characteristics of mesenchymal stem cells.

2) Cell surface marker detection

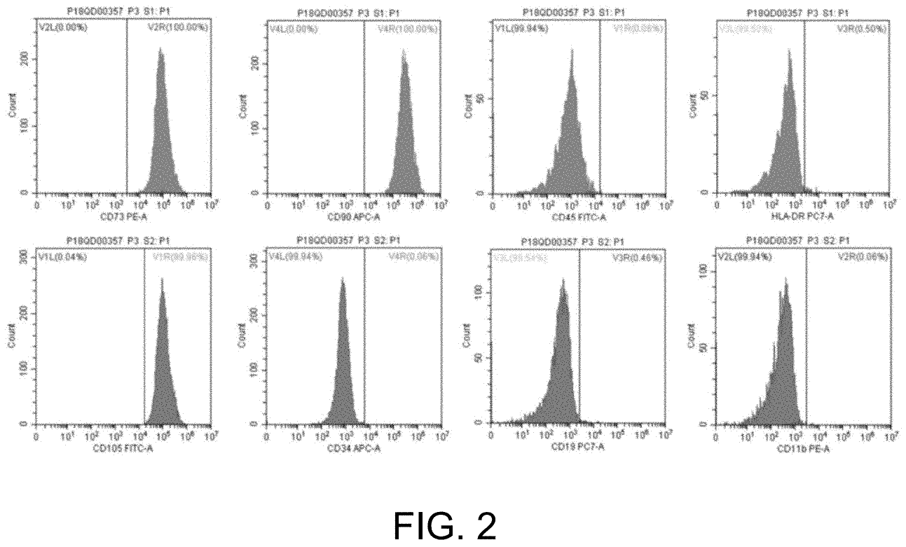

Cultured umbilical cord mesenchymal stem cells were used to detect the expression of positive markers (CD73, CD105, CD90) and negative markers (CD11b, CD19, CD34, CD45, HLA-DR) of MSCs by flow cytometry, according to international cell therapy. The Institute of Mesenchymal and Tissue Stem Cell Committee proposed a positive marker expression rate of ≥95% and a negative marker of ≤2%. The detection results were identified. CD73 expression was 100.0%, CD90 expression was 100.0%, CD105 expression was 99.96%, CD11b expression was 0.06%, CD19 expression was 0.46%, CD34 expression was 0.06%, CD45 expression was 0.6%, and the expression level of HLA-DR was 0.50%, which was consistent with the identification requirements of mesenchymal stem cells (FIG. 2).

3) Cell-induced differentiation potential detection

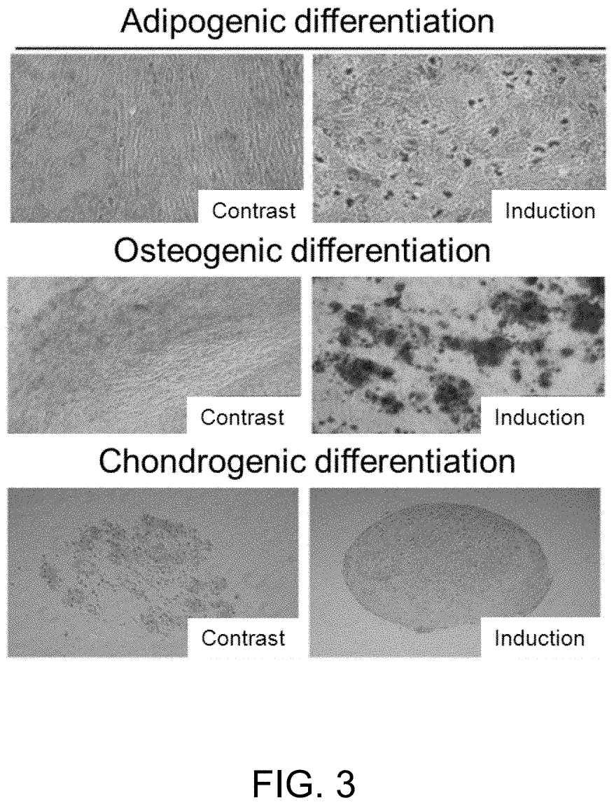

The umbilical cord mesenchymal stem cells in the logarithmic growth phase were induced by the adipogenic, osteogenic and chondrogenic differentiation medium for 3-4 weeks, and the differentiation potential was detected. The cells were identified by lipid red staining with oil red O. Compared with the control group, the induced umbilical cord mesenchymal stem cells showed small lipid droplets of different sizes after staining, indicating that umbilical cord mesenchymal stem cells have adipogenic differentiation potential; the alizarin red staining solution was used to identify the cells by osteogenic staining. Compared with the control group, the induced cells formed calcium nodules, which were stained dark red by alizarin red, indicating that umbilical cord mesenchymal stem cells have osteogenic differentiation potential; the cells were stained with chondrocytes by wood-eosin staining. And compared with the control group, the extracellular matrix of the induced group was stained deeply, indicating that umbilical cord mesenchymal stem cells have the potential for chondrogenic differentiation (FIG. 3).

When umbilical cord mesenchymal stem cells are cultured to the P3-P7 generation, they can be used in the preparation of stem cell preparations described herein.

The present invention is further illustrated below in conjunction with the embodiments:

### EXAMPLE 1

Formulations were prepared according to Table 1:

TABLE 1
Composition formulation

| | | | | | | | | Control | Control | | Control |
|---|---|---|---|---|---|---|---|---|---|---|---|
| | | Example 1 | | Example 1 | | Example 1 | | group 1 | group 2 | | group 3 |
| Solvent | Plasmalyte A | 350 | mL | 450 | mL | 500 | mL | FBS 95 mL | FBS 90 mL | | 200 mL |
| | DMSO | 100 | mL | 50 | mL | 50 | mL | 5 mL | 10 | mL | 100 mL |
| | 5 wt % | 500 | mL | 500 | mL | 350 | mL | — | — | | 200 mL |
| | glucose | | | | | | | | | | |
| | injection | | | | | | | | | | |
| Solute | Human | 150 | mg/mL | 100 | mg/mL | 10 | mg/mL | — | — | | 20 wt % |
| | serum | | | | | | | | | | 150 mL |
| | albumin | | | | | | | | | | |
| | PACAP | 100 | nmol/L | 1 | μmol/L | 10 | μmol/L | — | 1 | μmol/L | — |

| | | | | | | | | | |
|---|---|---|---|---|---|---|---|---|---|
| Vitamin C | 100 | μg/mL | 50 | μg/mL | 10 | μg/mL | — | — | — |
| Vitamin E | 1 | mg/mL | 5 | mg/mL | 10 | mg/mL | — | — | — |

The preparation method of the composition includes the following steps: mixing human serum albumin, plasmalyte A injection, clinical grade DMSO, glucose injection, PACAP, vitamin C, and vitamin E as a composition.

### EXAMPLE 2

The composition of each group in example 1 and the P5 umbilical cord mesenchymal stem cells were separately taken to prepare a stem cell preparation:

1) When the umbilical cord mesenchymal stem cell confluence is as long as 80%-90%, the culture medium in the culture dish is discarded, and washed with physiological saline for 2-3 times;

2) Add 3-5 mL of 0.05% trypsin for 1-2 min, observe the digestion under the microscope, and terminate the digestion with the serum-containing cell culture medium, and collect the cell suspension into the centrifuge tube;

3) Centrifuge at 500 g for 5 min under room temperature conditions, discard the supernatant after centrifugation, add plasmalyte A injection and mix thoroughly, sample and count;

4) According to the counting result, the cell density is adjusted to be 5×10⁶ cells/mL to 50×10⁶ cells/mL by using plasmalyte A injection;

5) Slowly add an equal volume of each group of the composition to the cell suspension respectively, mix and disperse into the cryotube, 0.5-2 ml per tube, label the cryotube and cool down according to the procedure, then transfer to store in liquid nitrogen. Among them, in the cell suspension to which the composition of the experimental group 1 was added, the final density of the cells was 2.5×10⁶ cells/mL; and in the cell suspension to which the composition of the experimental group 2 was added, the final density of the cells was 15×10⁶ cells/mL; and in the cell suspension to which the composition of the experimental group 3 was added, the final density of the cells was 25×10⁶ cells/mL; and in the cell suspension to which the composition of the control group 1 was added, the final density of the cells was 15×10⁶ cells/mL; and in the cell suspension to which the composition of the control group 2 was added, the final density of the cells was 15×10⁶ cells/mL; and in the cell suspension to which composition of the control group 3 was added, the final density was 15×10⁶ cells/mL.

### EXAMPLE 3

Umbilical cord mesenchymal stem cell preparations prepared from the experimental group and the control group were stored in liquid nitrogen for 1 month, 3 months and 6 months, respectively. After recovery in a 37° C. water bath, cell viability was detected by trypan blue staining. The results are shown in Table 2:

TABLE 2
Cell viability in different groups

| | | | | Control | Control | Control |
|---|---|---|---|---|---|---|
| | Example 1 | Example 2 | Example 3 | group 1 | group 2 | group 3 |
| 0 month | 97.72 ± 0.34 | 98.23 ± 0.21 | 98.45 ± 0.34 | 98.12 ± 0.36 | 98.05 ± 0.23 | 98.56 ± 0.64 |
| 1 month | 95.43 ± 0.42 | 97.49 ± 0.19 | 96.32 ± 0.64 | 93.26 ± 0.46 | 94.84 ± 0.35 | 92.27 ± 0.31 |
| 3 months | 94.35 ± 0.78 | 96.08 ± 0.45 | 94.28 ± 0.38 | 89.27 ± 0.17 | 90.44 ± 0.42 | 88.45 ± 0.44 |
| 6 months | 92.17 ± 0.64 | 95.72 ± 0.28 | 91.93 ± 0.72 | 85.73 ± 0.28 | 86.91 ± 0.11 | 85.11 ± 0.61 |

The results showed that the cell preparation of the example still maintained the cell viability well within 6 months, while the cell preparation in the comparative example decreased to below 90% after 3 months, indicating the composition formulation of the present patent can stably maintain the viability of umbilical cord mesenchymal stem cells, and the effect of each example was superior to each of the comparative examples (p<0.05). Among them, the cell viability of the experimental group 2 was the highest, and there was a significant difference (p<0.05) with the other groups.

### EXAMPLE 4

The efficacy of umbilical cord mesenchymal stem cell preparation was evaluated in animal model of OA

1) Cartilage injury (CI) model of New Zealand rabbit

The rabbit's hind legs were depilated and sterilized with 75% alcohol. The patella was incised from the side ring and moved to the side. The knee joint was bent 90° to expose the non-weight-bearing area of the medial femoral condyle. A pointed cone was then used to mark the position of the anterior upper end of the distal interstitial groove of the femur at a position of 4 mm, and the drill bit was used to make a cartilage defect of 3 mm in diameter and 1 mm in depth. The patella was then repositioned. After cleaning the wound, 2-0 vicryl absorbable suture was used to repair the joint cavity, and 3-0 mercerized suture was used to close the skin, and finally the skin was cleaned and trimmed.

A mixture of gentamicin (2 mg/kg) and ceftriaxone (50 mg/kg) was administered postoperatively for 24 hours at intervals of five days.

One week after the operation, the model animal joints were evaluated, and the animal model without swelling and obvious inflammatory response and with normal basic vital signs was selected for the follow-up efficacy evaluation experiment of stem cell preparation.

2) Evaluation of the efficacy of stem cell preparations for OA

The cartilage repair experiment of the rabbit cartilage injury model caused by OA was carried out by using the stem cell preparation prepared by the composition of embodiment 2, and the experiment was divided into a low dose group of stem cell preparation, a high dose group of stem cell preparation, a solvent control group and a model control group.

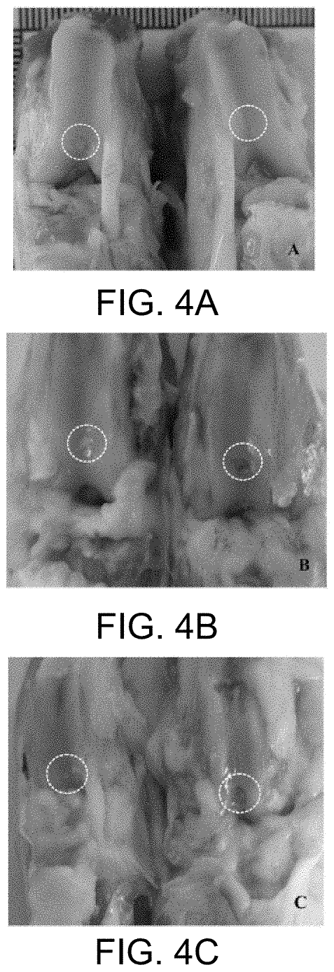

The result showed in FIG. 4A to FIG. 4C:

In the low dose (2×10⁶ cells) group (n=8), the cartilage repair rate was 62.5% (5/8).

In the high-dose (6×10⁶ cells) group (n=8), the cartilage repair rate was 75% (6/8).

In the solvent control group (n=8), the cartilage repair rate was 12.5% (1/8).

In the model control group (n=8), the cartilage repair rate was 12.5% (1/8).

The repair areas of cartilage in each group are shown in Table 3:

TABLE 3
Area of cartilage injury (mm²)

| | | | Solvent | Model |
|---|---|---|---|---|
| | Low dose | High dose | control | control |
| | group | group | group | group |
| Area of | 7.07 | 7.07 | 7.07 | 7.07 |
| cartilage injury | | | | |
| before treatment | | | | |
| Area of | 1.92 ± | 1.46 ± | 6.56 ± | 6.83 ± |
| cartilage injury | 0.10** | 0.37** | 0.37 | 0.23 |
| after treatment | | | | |

Note:
**showed a significant difference compared with that before treatment, p < 0.01.

The results showed that the umbilical cord mesenchymal stem cell preparation provided by the invention had a good repair effect on cartilage injury, and the repair area was significantly higher than that of the model group and the solvent control group (p<0.01), and the effect was dose-dependent.

The above is only the preferred mode of implementation of the invention, and it should be pointed out that for ordinary technicians in the technical field, some improvements and retouching can be made without deviating from the principle of the invention, and these improvements and retouching should also be considered as the protection scope of the invention.

1. A composition, comprising:
10 mg/mL to 150 mg/mL albumin;
100 nmol/L to 10 μmol/L pituitary adenylate cyclase activating polypeptide (PACAP);
10 μg/mL to 100 μg/mL vitamin C;
1 mg/mL to 10 mg/mL of vitamin E; and
solvents,
wherein the solvents consist of DMSO, plasmalyte A and 5 wt % glucose injection.

2. The composition according to claim 1, wherein a volume ratio of DMSO, plasmalyte A and 5 wt % glucose injection in the solvents is 5-10:35-50:35-50.

3. The composition according to claim 1, comprising:
100 mg/mL albumin;
1 μmol/L PACAP;
50 μg/mL vitamin C;
5 mg/mL vitamin E; and
solvents,
wherein the volume ratio of DMSO, plasmalyte A and 5 wt % glucose injection in the solvents is 5:45:50.

4. A cell cryopreservation solution, comprising the composition according to claim 1.

5. A method for preparing a stem cell preparation, comprising: using the composition according to claim 1.

6. A stem cell preparation, comprising:
mesenchymal stem cells; and
the composition according to claim 1.

7. The stem cell preparation according to claim 6, wherein the mesenchymal stem cells are umbilical cord mesenchymal stem cells, and a density of the mesenchymal stem cells is 2.5×10⁶ cells/mL to 25×10⁶ cells/mL.

8. The stem cell preparation according to claim 6, further comprising:
plasmalyte A, wherein a volume ratio of the plasmalyte A and the composition is 1:1.

9. A method for preparing a medicament for repairing cartilage injury, comprising: using the stem cell preparation according to claim 6.

10. A method for preparing a medicament for treating osteoarthritis, comprising: using the stem cell preparation according to claim 6.
